(19) United States
(12) Patent Application Publication
(10) Pub. No.: US 2019/0046744 A1
(43) Pub. Date: Feb. 14, 2019

(54) Fluid Delivery Apparatus

(71) Applicant: Sinaclear IP Pty Ltd, Paddington, Queensland (AU)

(72) Inventors: Alexandra Grace FRENCH, Milton (AU); Maddison Jane ROGERS, Milton (AU); Luke John CECCATO, Milton (AU)

(21) Appl. No.: 16/077,472

(22) PCT Filed: Feb. 12, 2016
(86) PCT No.: PCT/AU2016/050093

§ 371 Date: Aug. 11, 2018

(30) Foreign Application Priority Data
Feb. 13, 2015 (AU) 2015900474

(51) Int. Cl. A61M 15/00; A61M 11/04; A61M 3/02; A61M 15/08

(52) U.S. Cl. CPC A61M 15/0038; A61M 11/042; A61M 3/02; A61M 15/08; A61M 2205/36; A61M 2205/3341; A61M 2202/06; A61M 2205/3368; A61M 2210/0618

(57) ABSTRACT

The present invention provides an apparatus for delivering a fluid to a subject. The apparatus includes: (a) a vessel fillable with an amount of water through an opening; (b) a cap for covering the opening and configured to store an additive to be released into the water; and (c) a fluid delivery attachment including a body having: (i) a cap engaging portion configured to rupture a portion of the cap in order to release the additive into the water; (ii) a fluid outlet; and, (iii) a passageway extending between the cap engaging portion and the fluid outlet, wherein, the cap engaging portion is engageable with the cap so as to allow fluid to flow between the vessel and the fluid outlet.

### PRIORITY DOCUMENTS

The present application claims priority from Australian Provisional Application No. 2015900474 titled “FLUID DELIVERY APPARATUS” and filed on 13 Feb. 2015, the content of which is hereby incorporated by reference in its entirety.

### BACKGROUND OF THE INVENTION

The present invention relates to an apparatus for delivering a fluid to a subject. In a particular form, the invention relates to a nasal irrigation apparatus for delivering an irrigation solution to a nasal cavity of the subject.

### DESCRIPTION OF THE PRIOR ART

The reference in this specification to any prior publication (or information derived from it), or to any matter which is known, is not, and should not be taken as an acknowledgment or admission or any form of suggestion that the prior publication (or information derived from it) or known matter forms part of the common general knowledge in the field of endeavour to which this specification relates.

Nasal irrigation or rinsing, also known as sinus irrigation or nasal lavage, is the practice of irrigating the nasal cavity of a subject with an irrigation fluid, typically a saline solution. There are many reasons why nasal irrigation is practiced including to reduce or prevent mucous build-up, moisten the nasal passage and to prevent or assist in treating sinus infections, rhinitis, postnasal drip and the like. It may also be useful as a post operative procedure to soften and remove crusting associated with sinus surgery and as a regular, preventative and proactive cleansing practice to maintain a clear nasal cavity free of mucous, dust, dirt, irritants and other infectious agents.

Various nasal irrigation devices have been developed. One such example is the neti pot which is a small pot with a spout that allows an irrigation solution to flow under gravity into the nasal cavity as a subject tilts their head to one side and then the other. Typically, boiling or lukewarm water is poured into the pot and mixed with a saline additive. Typically, to mix the water and saline additive together, a lid is screwed onto the top of the pot and the subject places a finger over the spout while the pot is shaken. Touching the spout in this manner is not desirable as the saline solution flows out of the spout and if the subject's hands are not clean, it is possible for bacteria or dirt to be transferred into the spout. The cap typically has a threaded connection to the pot which creates surfaces and grooves on both the cap and the pot that can be difficult to properly clean and that therefore may collect bacteria over time through repeated use. The pot itself is typically cleaned with hot water. However some internal surfaces, including of the spout for example, can be difficult to access and thoroughly clean. Accordingly, the neti pot, while simple to use, suffers from having too many surfaces on which bacterial colonies may grow.

Other devices deliver the irrigation solution under a positive pressure. For example, there are squeeze bottles containing the solution that can be squeezed to pump the solution through a nasal adaptor and into the nasal cavity. More sophisticated positive pressure devices use an electric motor driven pump.

Positive pressure devices typically include a flexible bottle and a nasal adaptor or cap that is screwed onto the top of the bottle. The adaptor usually has a tube that extends into the bottle through which the solution flows when the bottle is squeezed. Similar to a neti pot, these devices are relatively simple to use, however provide many potential bacteria harbouring surfaces. For example, the nasal adaptor or cap typically includes a threaded portion that is screwed onto a mating thread on the bottle. As discussed above, threaded regions are difficult to clean properly and are therefore regions likely to harbour bacteria. When a squeeze bottle is shaken to mix the water and saline additive together, a subject typically puts a finger over the nasal adaptor to prevent any of the solution from spilling. Physical contact with the fluid outlet is not desirable as bacteria can easily be passed by this action. Finally, in regards to cleaning, the tube of a squeeze bottle is an area difficult to access and clean thoroughly which can lead to bacterial growth inside the tube which is the primary fluid passage of the device.

The long term sterility of existing nasal irrigation devices is therefore a concern for the above mentioned reasons. Currently, the best method of reducing the likelihood of bacterial infection from use of these devices is to replace them altogether at time varying intervals. This can be costly and inconvenient depending on how often the devices need to replaced.

A further problem with existing devices is that there is no control over what irrigation solution is used. A subject may elect to use whatever rinsing fluid or mixture they like for the irrigation. While subjects are able to purchase specific nasal rinse sachets usually containing a particular mix of sodium chloride and a suitable buffering agent such as sodium bicarbonate, often subjects prefer to create their own solution at home. The risk associated with this is that undesirable additives may be introduced into the irrigation solution, particularly if regular table or sea salt is used. Further it can be difficult to gauge the quantity of ingredients being added to the solution which can lead to solutions having unknown tonicities.

Even if nasal rinse sachets are purchased and added to the solution, this is still a manual process requiring the subject to tear the sachet and empty the contents into the water to be mixed.

While the above discussion has focused in particular on nasal irrigation devices, it is to be appreciated that similar problems exist for steam inhalation devices that involve heating water (or using boiled water) to produce steam that a subject inhales through their nose and mouth in order to ease mucous production, nasal congestion, throat discomfort, coughing and the like. Such devices may be used with fragrant additives, such as menthol or eucalyptus oil which assist in creating a soothing vapour. As with nasal irrigation devices, the sterility of steam inhalation devices is important to prevent bacteria from growing on surfaces of the device that can lead to contaminated steam and airborne allergens being inhaled into the body.

It is against this background, and the problems and difficulties associated therewith, that the present invention has been developed.

Accordingly, it would be desirable to provide an apparatus for delivering fluid to a subject, such as a nasal irrigation or inhalation apparatus that has increased sterility by reducing susceptibility to bacterial growth.

Other objects and advantages of the present invention will become apparent from the following description, taken in connection with the accompanying drawings, wherein, by way of illustration and example, an embodiment of the present invention is disclosed.

### SUMMARY OF THE PRESENT INVENTION

In one broad form the present invention seeks to provide an apparatus for delivering a fluid to a subject, the apparatus including:
a) a vessel fillable with an amount of water through an opening;
b) a cap for covering the opening and configured to store an additive to be released into the water; and
c) a fluid delivery attachment including a body having:
   i) a cap engaging portion configured to rupture a portion of the cap in order to release the additive into the water;
   ii) a fluid outlet; and
   iii) a passageway extending between the cap engaging portion and the fluid outlet,
wherein, the cap engaging portion is engageable with the cap so as to allow fluid to flow between the vessel and the fluid outlet.

Typically the cap includes a housing having a top portion containing a weakened region.

Typically the additive is stored below the top portion.

Typically the additive is stored in the cap by a seal configured to hold the additive.

Typically at least part of the cap engaging portion is configured to be inserted into the cap so as to rupture a part of the top portion in the vicinity of the weakened region.

Typically rupturing the part of the top portion causes the seal to be ruptured to thereby release the additive into the water.

Typically the part of the top portion of the cap that is ruptured remains hingedly connected to the cap allowing it to fold out of the way as the at least part of the cap engaging portion is inserted into the cap.

Typically the seal remains hingedly connected to the cap after it has been ruptured, the seal hinging about the same location as the part of the top portion of the cap that is ruptured.

Typically the cap engaging portion includes a tubular element that terminates in a longitudinal end having a cutting portion configured to rupture the part of the top portion.

Typically the tubular element is a split tube configured so that a part of the weakened region remains intact when the cutting portion ruptures the part of the top portion.

Typically the vessel includes a body having a neck region around the opening.

Typically the cap housing includes an outer wall spaced apart from an inner wall that both downwardly depend from the top portion.

Typically the neck region of the vessel is receivable between the inner wall and the outer wall to thereby locate the inner wall within the opening of the vessel.

Typically the tubular element is positionable within the inner wall to thereby allow fluid to flow from the vessel into the fluid delivery attachment.

Typically the tubular element includes one or more resiliently deformable tabs engageable with a bottom surface of the inner wall so as to retain the cap with respect to the fluid delivery attachment.

Typically the cap housing further includes a grippable release element that is able to be pulled in order to release or tear the cap away from the fluid delivery attachment.

Typically the grippable release element is attached to the outer wall.

Typically the grippable release element is a flexible tab.

Typically the cap is for a single use.

Typically the fluid delivery attachment is releasably engageable with the vessel.

Typically the vessel is made from glass or a non-porous plastic.

Typically the vessel includes an elastomeric one-way valve which selectively allows air into the vessel in order to balance air pressure inside and outside of the vessel in order to regulate flow of fluid through the fluid outlet.

Typically the fluid delivery attachment is made from a non-porous plastic.

Typically the additive is a dissolvable tablet.

Typically the additive is a liquid.

Typically the additive contains a saline composition.

Typically the additive contains a fragrance or medicament.

Typically the fluid delivery attachment is for nasal irrigation.

Typically the fluid delivery attachment is for steam inhalation.

Typically the apparatus further includes a base portion configured to receive the vessel.

Typically the base portion is configured to heat the vessel.

Typically the base portion includes one or more inductive heating elements.

Typically the cap includes a housing having a first seal located at the top of housing and second seal located at the bottom of the housing, and wherein the additive is stored in a cavity between the first and second seals.

Typically at least part of the cap engaging portion is configured to be inserted into the cap so as to rupture a part of the first and second seals to thereby cause the additive to be released into the water.

Typically the vessel is a two-part construction including a body portion releasably engageable with a lid portion, the opening disposed in the lid portion.

In another broad form the present invention seeks to provide a nasal irrigation apparatus for irrigating a nasal cavity of a subject, the apparatus including:
a) a vessel fillable with an amount of water through an opening;
b) a cap for covering the opening and configured to store an additive to be released into the water; and
c) an irrigation attachment, including a body having:
   i) a cap engaging portion configured to rupture a portion of the cap in order to release the additive into the water to form an irrigation fluid;
   ii) a nasal adaptor portion configured to be inserted into a nostril of the subject, said nasal adaptor portion terminating in a fluid outlet for expelling said irrigation fluid into the nasal cavity; and
   iii) a passageway extending between the cap engaging portion and the fluid outlet,
wherein, the cap engaging portion is engageable with the cap so as to allow irrigation fluid to flow between the vessel and the fluid outlet.

In another broad form the present invention seeks to provide a steam inhalation apparatus for directing steam into a nasal cavity of a subject, the apparatus including:
a) a vessel fillable with an amount of water through an opening;
b) a cap for covering the opening and configured to store an additive to be released into the water; and
c) an inhalation attachment, including a body having:
   i) a cap engaging portion configured to rupture a portion of the cap in order to release the additive into the water; and
   ii) a mask portion configured to cover a portion of the subject's face including a nose and mouth of the subject, the mask portion including a steam outlet for directing steam into the nasal cavity; and
   iii) a passageway extending between the cap engaging portion and the steam outlet,
wherein, the cap engaging portion is engageable with the cap so as to allow steam to flow between the vessel and the steam outlet when the water is vapourised.

In another broad form the present invention seeks to provide a cap for releasing an additive into an amount of water in a vessel, the cap including:
a) a housing engageable with the vessel to cover an opening thereof, the housing configured to store the additive, the housing including:
   i) a rupturable portion configured to be ruptured when a fluid delivery attachment for delivering fluid to a subject is engaged with the housing to thereby cause the additive to be released into the water,
   wherein, the rupturable portion is rupturable so as to allow at least part of the fluid delivery attachment to be inserted into the housing in the vicinity of the opening of the vessel to thereby allow fluid to flow from the vessel to the fluid delivery attachment whereby it is deliverable to the subject.

Typically the rupturable portion contains a weakened region configured to be ruptured by the fluid delivery attachment.

Typically the additive is stored below the rupturable portion.

Typically the additive is held in the housing by a seal.

Typically rupturing the rupturable portion causes the seal to be ruptured to thereby release the additive into the water.

Typically the rupturable portion remains hingedly connected to the housing after it has been ruptured so as to allow it to fold out of the way as the at least part of the fluid delivery attachment is inserted into the housing.

Typically the seal remains hingedly connected to the housing after it has been ruptured, the seal hinging about the same location as the rupturable portion.

Typically the housing includes:
a) an inner wall; and
b) an outer wall spaced apart from the inner wall to thereby define an opening receivable of a neck region of the vessel.

Typically the housing further includes a top portion from which the inner wall and the outer wall downwardly depend.

Typically at least part of the top portion forms the rupturable portion.

Typically the housing further includes a grippable release element that is able to be pulled in order to release or tear the cap away from the fluid delivery attachment.

Typically the grippable release element is attached to the outer wall.

Typically the grippable release element is a flexible tab.

Typically the cap is for a single use.

Typically the additive includes at least one of:
a) a saline composition;
b) a medicament; and
c) a fragrance.

Typically the additive is either in solid or liquid form.

Typically the rupturable portion includes a first seal located at the top of the cap housing and second seal located at the bottom of the cap housing, the additive stored in a cavity between the first and second seals.

Typically the first and second seals are plastic films.

### BRIEF DESCRIPTION OF THE DRAWINGS

An example of the present invention will now be described with reference to the accompanying drawings, in which:

FIG. 1A is a cross sectional view of an example of an apparatus for delivering a fluid to a subject;

FIG. 1B is a cross sectional view of the apparatus of FIG. 1A showing a fluid delivery attachment rupturing a cap attached to a vessel;

FIG. 1C is a cross sectional view of the apparatus of FIG. 1A showing the apparatus in an assembled configuration, when additive is released into the vessel.

FIG. 2A is a side view of the apparatus of FIG. 1 in an assembled configuration;

FIG. 2B is a cross sectional view taken along section A-A of FIG. 2A showing the fluid delivery attachment attached to the vessel and cap.

FIG. 3 is a cross sectional view of the apparatus of FIG. 1A showing disengagement of the fluid delivery attachment from the vessel;

FIG. 4 is a cross sectional view of an example of a cap for covering an opening of the vessel;

FIG. 5A is a cross sectional view of the cap of FIG. 4 about to be engaged with the vessel;

FIG. 5B is a cross sectional view of the cap of FIG. 4 engaged with the vessel;

FIG. 6 is a detailed sectional perspective view of the fluid delivery attachment about to rupture the cap attached to the vessel;

FIG. 7 is a bottom view of the fluid delivery attachment;

FIG. 8A is a rear view of the apparatus of FIG. 1 in an assembled configuration;

FIG. 8B is a cross sectional view taken along section B-B of FIG. 8A showing the engagement of the fluid delivery attachment to the cap;

FIG. 9A is a front view of a further example of an apparatus for delivering a fluid to a subject having an alternative fluid delivery attachment;

FIG. 9B is a side view of the apparatus of FIG. 9A;

FIG. 10 is a schematic view of an example of a subject using the apparatus of FIG. 1 to irrigate a nasal cavity of the subject;

FIG. 11A is a perspective view of an example of a two-part vessel forming part of an apparatus for delivering a fluid to a subject;

FIG. 11B is an exploded view of the vessel shown in FIG. 11A;

FIGS. 11C and 11D show perspective views of an example of a cap engaging with the vessel of FIG. 11A;

FIG. 11E is a cross sectional view through the vessel and cap shown in FIG. 11D showing the top and bottom seals of the cap;

FIGS. 11F to 111 provide perspective and side views of an apparatus for delivering fluid to a subject showing engagement of an example of a fluid delivery attachment to the vessel shown in FIG. 11A; and;

FIG. 11J is a cross sectional view of an example of the apparatus showing the cap engaging portion of the fluid delivery attachment rupturing the cap to thereby release the additive into the vessel.

### DETAILED DESCRIPTION OF THE PREFERRED EMBODIMENTS

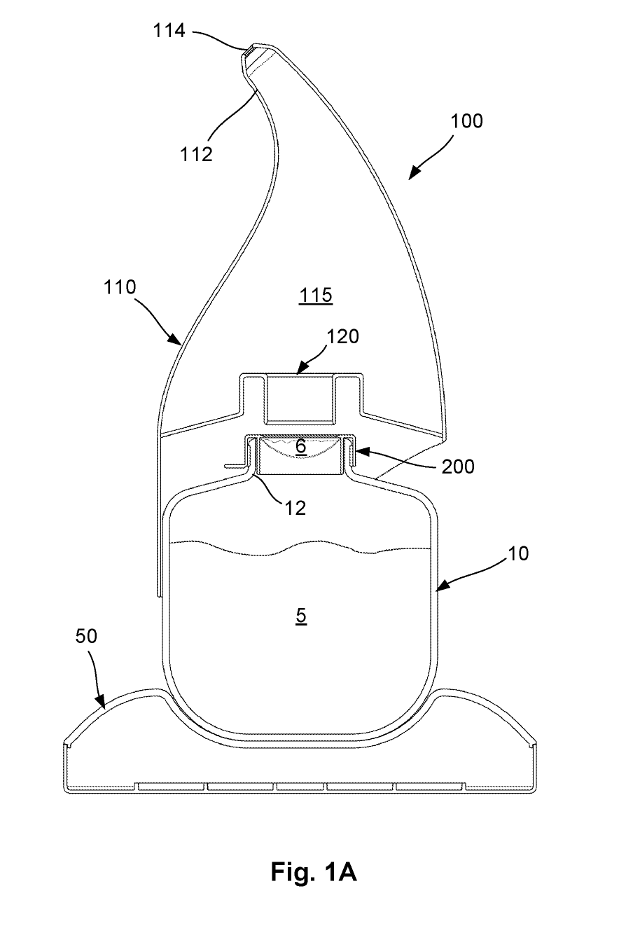

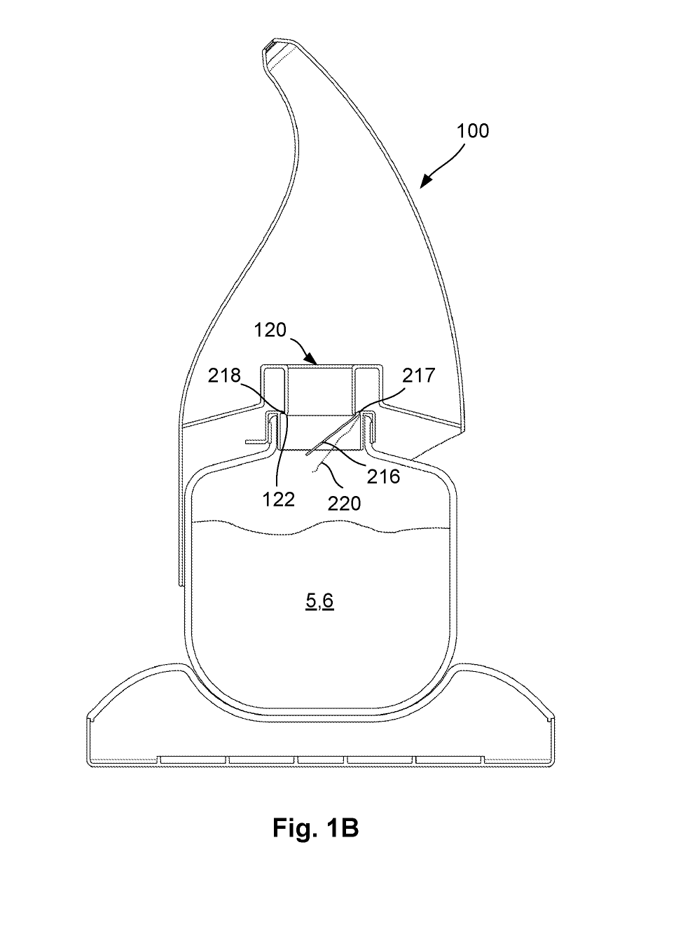

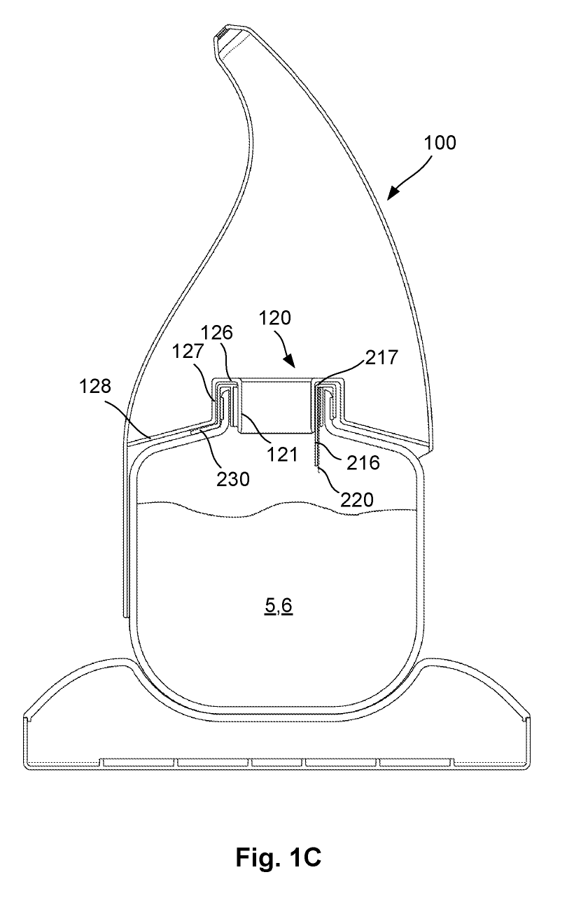

An example of an apparatus 100 for delivering a fluid to a subject will now be described with reference to FIGS. 1A to 1C. The subject, whilst ordinarily representing a person, is not limited to such and may be taken to also include certain animals such as dogs and horses. It is also to be understood that the term “fluid” as used in this specification may include either a liquid or a gas, for example water vapour or steam.

In this example, the apparatus 100 includes a vessel 10 fillable with an amount of water 5 through an opening 12. The vessel 10 is preferably made from glass, such as quartz glass or borosilicate glass. Alternatively, the vessel 10 may be made from a non-porous plastic including for example polycarbonate or another suitable type of medical grade plastic. Furthermore, thermoplastic materials may be used with colour changing properties which may be responsive to temperature for example in order to indicate when fluid in the vessel is at a suitable temperature for use.

The apparatus 100 further includes a cap 200 for covering the opening 12 and configured to store an additive 6 to be released into the water 5. The additive used will be dependent on the application for which the apparatus 100 is used. For example, when the apparatus 100 is used for nasal irrigation, the additive will include a saline composition typically comprising sodium chloride and a suitable buffering agent such as sodium bicarbonate. When the apparatus 100 is used for steam inhalation, the additive may include an aromatic fragrance such as eucalyptus, menthol, tea tree, rosemary, pine, cedarwood, lavender and the like. Alternatively, the additive may include a medicament such as an antihistamine, corticosteroid, anti-inflammatory, anti-biotic etc. The additive 6 may be provided in solid form as a dissolvable tablet or powder or in liquid form such as a saline solution for nasal irrigation or an essential oil for steam inhalation.

A fluid delivery attachment 110 is used to delivery fluid from the vessel to the subject. The fluid delivery attachment 110 includes a body having a cap engaging portion 120 configured to rupture a portion of the cap 200 in order to release the additive 6 into the water 5. The attachment 110 further includes a fluid outlet 114 and a passageway 115 extending between the cap engaging portion 120 and the fluid outlet 114. As shown in FIGS. 1B and 1C, the cap engaging portion 120 is engageable with the cap 200 so as to allow fluid to flow between the vessel 10 and the fluid outlet 114.

It will be appreciated from the foregoing that after the cap 200 has been ruptured by the cap engaging portion 120 of the fluid delivery attachment 110 so as to release the additive 6, the cap is rendered non-reusable by the user thereby making it a single use cap. Consequently, a new cap is required for each subsequent use of the apparatus 100. It may be possible however to provide a two-part cap containing both an inhalation solution and an irrigation solution that can be selectively chosen by the user depending on whether a steam inhalation or nasal irrigation is required.

The above described apparatus 100 therefore reduces the risk of bacterial build up by preventing the cap 200 from being used more than once. Furthermore, the additive 6 is released into the vessel 10 filled with water 5 while the opening 12 of the vessel 10 is covered by the cap 200 and fluid delivery attachment 110. The apparatus 100 can then be shaken in order to mix the additive 6 with the water 5 without having to cover any outlet or opening with a finger or the like. The apparatus 100 provided is therefore a more hygienic device which reduces the likelihood of bacterial growth on any surfaces on any surfaces thereof. These advantages are increased further by the materials used for the reusable vessel and fluid delivery attachment which are non-porous materials that minimise the surfaces on which bacteria is able to grow. As a result of minimising the risk of bacterial growth, the lifespan of the apparatus 100 may be increased leading to a significant cost benefit to persons that frequently use such an apparatus 100.

A further advantage of the apparatus 100 is that the additive 6 is stored in the cap so the quality and composition of the additive 6 is able to thereby be controlled for each use of the apparatus 100. This eliminates the risk of any potentially harmful compositions being used.

A number of further features will now be described.

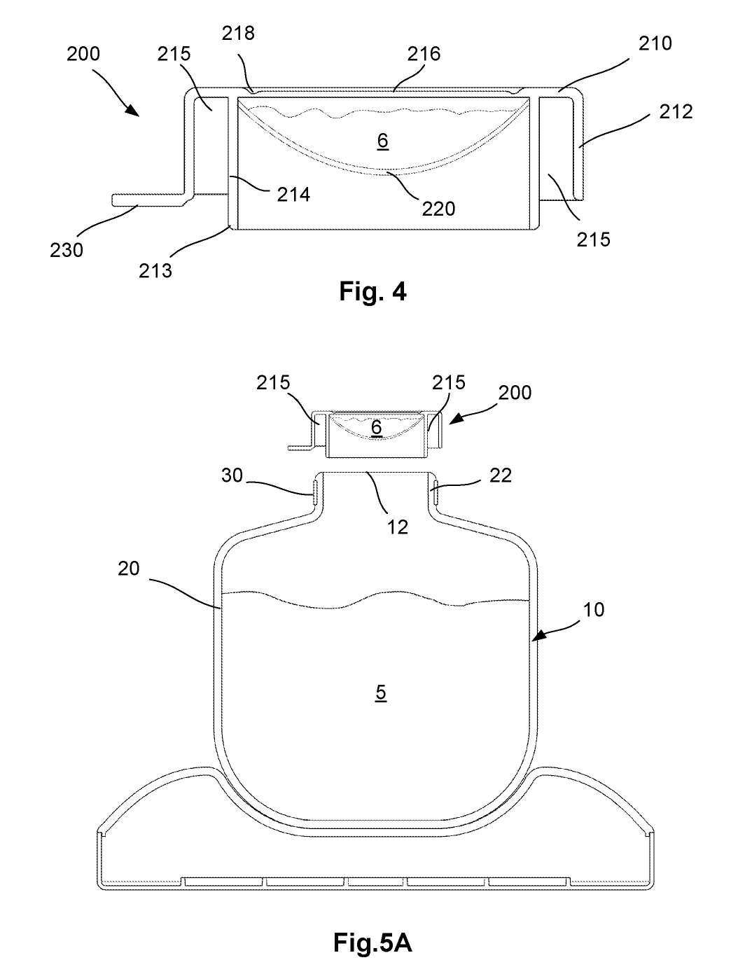

An example of a cap 200 suitable to be used with the apparatus 100 shown in FIGS. 1A to 1C, for releasing an additive 6 into an amount of water in a vessel 10 is shown in more detail in FIG. 4. The cap 200 includes a housing 210 engageable with the vessel 10 to cover an opening 12 thereof. The housing 210 is configured to store the additive 6. The housing 210 includes a rupturable portion 216 configured to be ruptured when a fluid delivery attachment for delivering fluid to a subject is engaged with the housing 210 to thereby cause the additive 6 to be released into the water 5. The rupturable portion 216 is rupturable so as to allow at least part of the fluid delivery attachment 110 to be inserted into the housing 210 in the vicinity of the opening 12 of the vessel 10 to allow fluid to flow from the vessel 10 to the fluid delivery attachment 110 whereby it is deliverable to a subject.

The rupturable portion 216 shown in FIG. 4 is a top portion of the housing 210. While in this example, the top portion 216 forms the rupturable portion of the housing 210, it is to be appreciated that this is not intended to be limiting. In other examples, the rupturable portion of the housing 210 may be disposed closer to the bottom of the housing, for example when the additive is stored in a lower region of the cap. In the following description, the rupturable portion of the housing 210 shall be described with reference to the top portion 216.

The top portion 216 contains a weakened region 218. The weakened region 218 may include for example a notch, groove, perforation, or other suitable element for the purpose of creating a region whereby the cap may easily be ruptured or pierced. The top portion 216 may also include a locally thinned region further providing an area of weakness.

Typically, the cap housing 210 will further include an outer wall 212 spaced apart from an inner wall 214. Both the inner wall 214 and the outer wall 212 downwardly depend from the top portion 216. It will be appreciated that the inner wall 214 and outer wall 212 of the housing 210 are generally cylindrical sections suitable for engaging with the vessel 10 in order to cover the opening 12. Accordingly, the top portion 216 and weakened region 218 will generally be circular. The cap housing 210 is typically made from a plastic such as polyethylene terephthalate (PET), polypropylene or any suitable medical grade plastic, although suitable metals such as steel or aluminium may also be used.

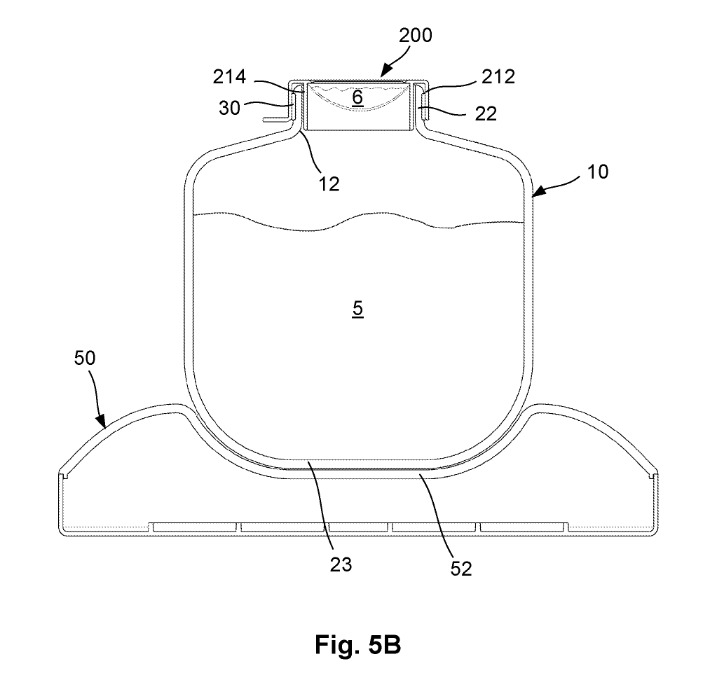

As shown in FIGS. 5A and 5B, the vessel 10 includes a body 20 having a neck region 22 around the opening 12 through which water is poured in order to fill the vessel 10 as required. A seal 30, such as an O-ring or other type of suitable rubber seal is typically located around the outer periphery of the neck region 22 in order to create a sealing engagement with the cap 200. The seal 30 may be removable or alternatively it may be overmoulded around the neck region 22. As shown in FIG. 5B, the neck region 22 of the vessel 10 is receivable between the inner wall 214 and the outer wall 212 to thereby locate the inner wall 214 within the opening 12 of the vessel 10. When the cap 200 is engaged with the vessel 10, the neck region 22 and seal 30 are sandwiched between the inner wall 214 and the outer wall 212 in channel 215.

Typically, the additive 6 is stored below the top portion 216 as shown for example in FIG. 4. The additive 6 is stored in the cap 200 by a seal 220 configured to hold the additive 6. The seal 220 is a frangible element preferably made from foil, although suitable plastic or rubber materials may also be used. In one example, the seal 220 is a pierceable foil heat seal that is thermally bonded to the cap 200. As shown in FIG. 4, the seal 220 may be bonded between portions of the inner wall 214 so as to form a sealed cavity between the top portion 216 and the seal 220.

As shown in FIG. 5B, the cap 200 is typically pressed onto the neck region 22 of the vessel 10 and is thereby held by frictional engagement. In this way, a threaded connection is avoided which is advantageous as threaded regions are difficult to clean and are areas where bacteria can grow.

The apparatus 100 may include a base portion 50 configured to receive the vessel 10. The base portion 50 may have a recessed portion 52 as shown in FIG. 5B that is contoured to receive a lower portion 23 of the vessel 10 therein. The base portion 50 provides a support for the apparatus 100 when not in use. The base portion 50 may further provide a convenient location to sit the vessel 10 while it cools down after it has been filled with boiled water.

Optionally, the base portion 50 may also be configured to heat the vessel 10. In one example, the base portion 50 may include one or more inductive heating elements that are operable to heat the vessel 10 by electrical induction. As the vessel 10 is typically made from glass or non-porous plastic, a ferromagnetic plate may be required to interface with the base of the vessel. Alternative electric heating elements may also be used including for example thick film heating elements. In examples where the apparatus 100 is operable to heat the vessel 10, selective temperature control may be provided to enable a user to heat the water inside the vessel 10 to a desired temperature.

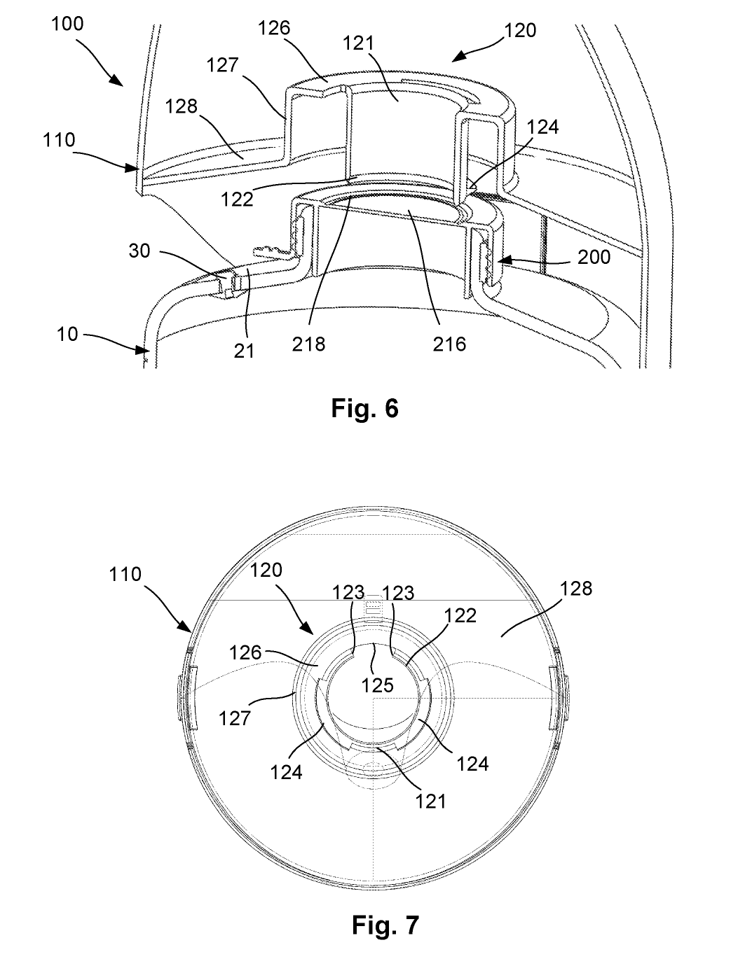

The cap engaging portion 120 will now be described in further detail with reference to FIGS. 6 and 7. The cap engaging portion 120 is configured to be at least partially inserted into the cap 200 so as to rupture a part of the top portion 216 in the vicinity of the weakened region 218. The cap engaging portion 120 typically includes a tubular element 121 that terminates in a longitudinal end having a cutting portion 122 configured to rupture the part of the top portion 216.

The cutting portion 122 may have a blade like edge formed by providing a chamfer or bevel to the end of the tubular element 121. This locally reduces the thickness at the end of the tubular element, for example to form a sharpened tip able to easily rupture the top portion 216 of the cap 200. The tubular element 121 is sized so that the cutting portion 122 has a diameter coincident with the weakened region 218 of the top portion 216 of the cap 200. While the tubular element 121 may be an enclosed cylindrical structure, it is preferred that the tubular element 121 is open so that the cutting portion 122 defines an open arc. As shown most clearly in FIG. 7, the tubular element 121 has an open cross section defining an opening 125 between ends 126. In other words, the tubular element 121 is a split tube configured so that a part of the weakened region 218 remains intact when the cutting portion 122 ruptures the part of the top portion 216. This will be discussed in further detail below.

The cap engaging portion 120 is typically attached to or integral with a wall of the fluid delivery attachment 110 as shown for example in FIG. 6. In this example, the cap engaging portion 120 includes an outer disc 128 removably attached or integral with the fluid delivery attachment 110. The disc 128 may have a complementary profile to an upper portion 21 of the vessel 10 on which it may be seated on or in close proximity to when cap engaging portion 120 is engaged with the cap 200.

In the example shown, the disc 128 extends between the wall of the fluid delivery attachment 110 and an internal flange 127 of the cap engaging portion 120. The internal flange 127 is generally cylindrical and configured to locate around the outer wall 212 of the cap 200 when the cap engaging portion 120 is engaged with the cap 200. The tubular element 121 is located within the internal flange 127 so as to be spaced apart therefrom. The tubular element 121 and flange 127 are indirectly coupled via top portion 126 from which the tubular element downwardly depends.

An example of the operation of the apparatus 100 shall now be described.

Firstly, a subject fills the vessel 10 with water to an appropriate fill line. For use in nasal irrigation, the water is usually sterilised or distilled or alternatively tap water that has been boiled in order to sterilise it. If the apparatus 100 permits heating then the water in the vessel 10 may be heated to a comfortable temperature for irrigation. Alternatively, the water may be boiled and allowed to cool down to a comfortable temperature. In the case of steam inhalation, boiling water is used or the apparatus may be configured to boil the water itself.

With the water 5 in the vessel 10, an appropriate cap 200 is selected depending on whether the apparatus is to be used for nasal irrigation or steam inhalation. As previously mentioned, for nasal irrigation, the cap 200 will contain an additive 6 containing a saline composition and/or medicament whereas for steam inhalation the cap 200 will contain an additive 6 containing an aromatic fragrance or medicament. The cap 200 is pressed onto the neck region 22 of the vessel 10 as shown in FIG. 5B whereby it is sealingly or seamingly engaged.

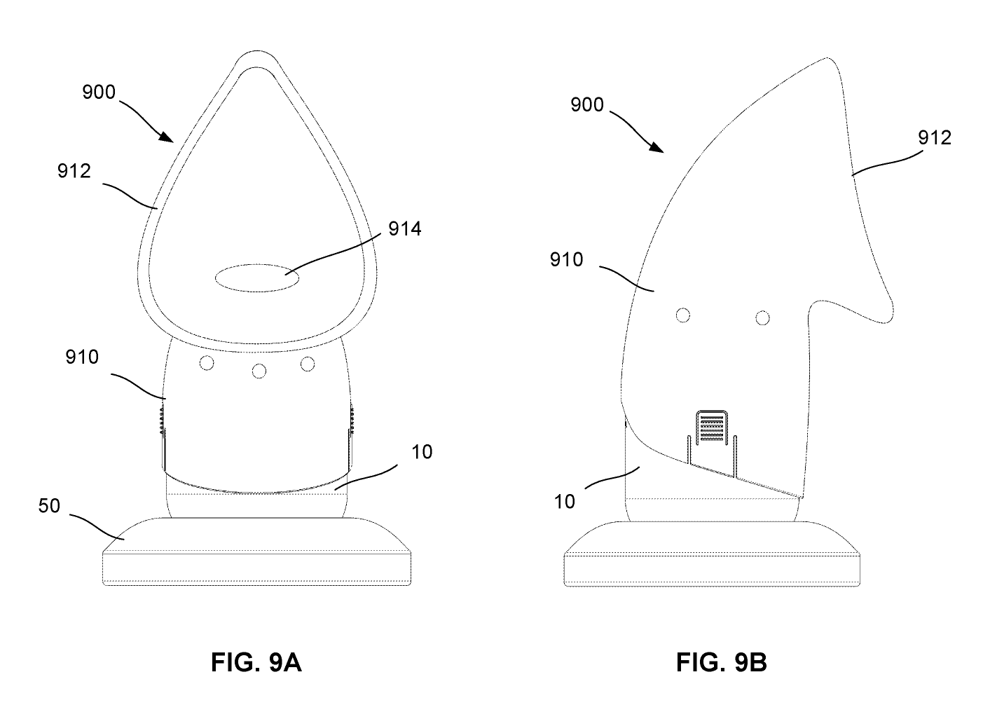

A suitable fluid delivery attachment is then selected based on whether a nasal irrigation or steam inhalation is being performed. The fluid delivery attachment shown in FIGS. 1A to 3, FIGS. 6 to 8B and FIG. 10 is for nasal irrigation while the fluid delivery attachment shown in FIGS. 9A to 9B is for steam inhalation as will be described in more detail later.

As shown in FIG. 1A, the fluid delivery attachment 110 is brought into alignment with the vessel 10 so that the tubular element 121 of the cap engaging portion 120 is aligned with the top portion 216 of the cap 200. The tubular element 121 is then brought into contact with the top portion 216 so that the cutting portion 122 is located within the weakened region 218. The fluid delivery attachment 110 is then urged firmly downwards so that the cutting portion 122 ruptures or pierces part of the top portion 216 in the vicinity of the weakened region 218.

As the tubular element 121 and cutting portion 122 define an open arcuate structure, a portion of the weakened region 218 is not ruptured as the tubular element 121 is inserted through the top portion 216. In this way, the part of the top portion 216 of the cap 200 that is ruptured remains hingedly connected to the cap 200 at hinge point 217 allowing it to fold out of the way as the cap engaging portion 120 is inserted into the cap 200. This prevents any portion of the ruptured cap from breaking off and falling into the water 5.

Rupturing the part of the top portion 216 causes the seal 220 to be ruptured or broken to thereby release the additive 6 into the water 5. In an example, as the top portion 216 ruptures and begins to fall away it will contact the seal 220, which when combined with the downward force of the tubular element 121 will break the thermal bond of the seal 220 with the inner wall 214 of the cap 200. Alternatively, the seal 220 may be ruptured or pierced at an intermediate location along its length. In the example shown in FIG. 1B, the seal 220 may also remain hingedly connected to the cap 200 after it has been ruptured, hinging about the same location as the part of the top portion 216 of the cap 200 that is ruptured.

As shown in FIG. 1C, when fully engaged, the tubular element 121 is disposed within the opening 12 of the vessel 10 and within the inner wall 214 of the cap 200 while the flange 127 is located around the periphery of the outer wall 212 of the cap 200. Top portion 126 of the cap engaging portion 120 is seated upon the remaining top portion 216 of the cap 200. The ruptured top portion 216 of the cap 200 and the seal 220 are folded out of the way about hinge point 217 to ensure that no undesirable fragments of the cap or seal break off into the water 5.

In the arrangement shown in FIG. 1C, the tubular element 121 is therefore positionable within the inner wall 214 of the cap 200 to thereby allow fluid to flow from the vessel 10 into the fluid delivery attachment 110. Tubular element 121 therefore forms a fluid passageway between the vessel 10 and fluid delivery attachment 110.

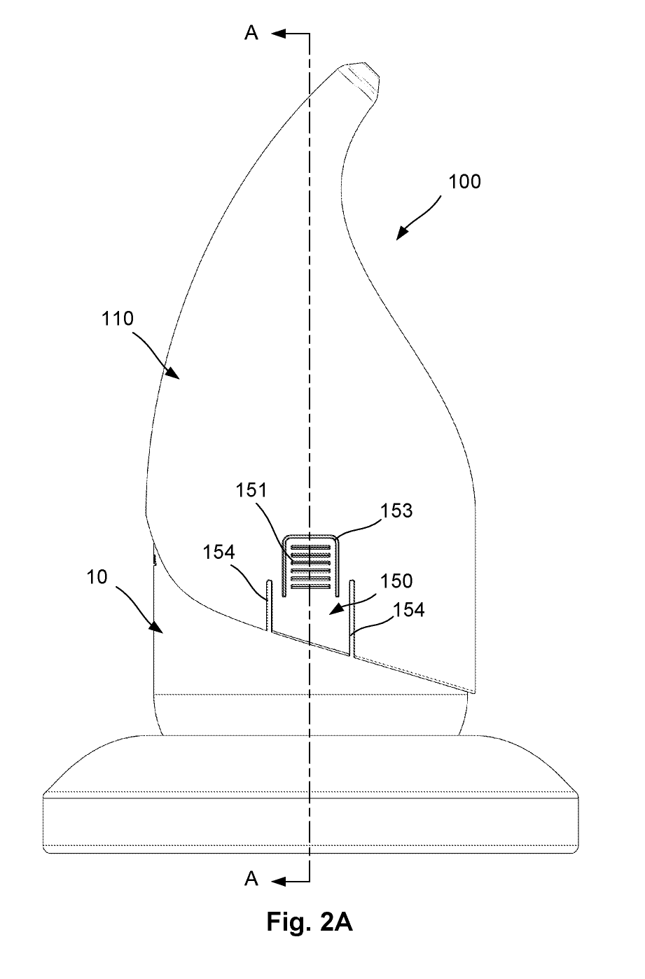

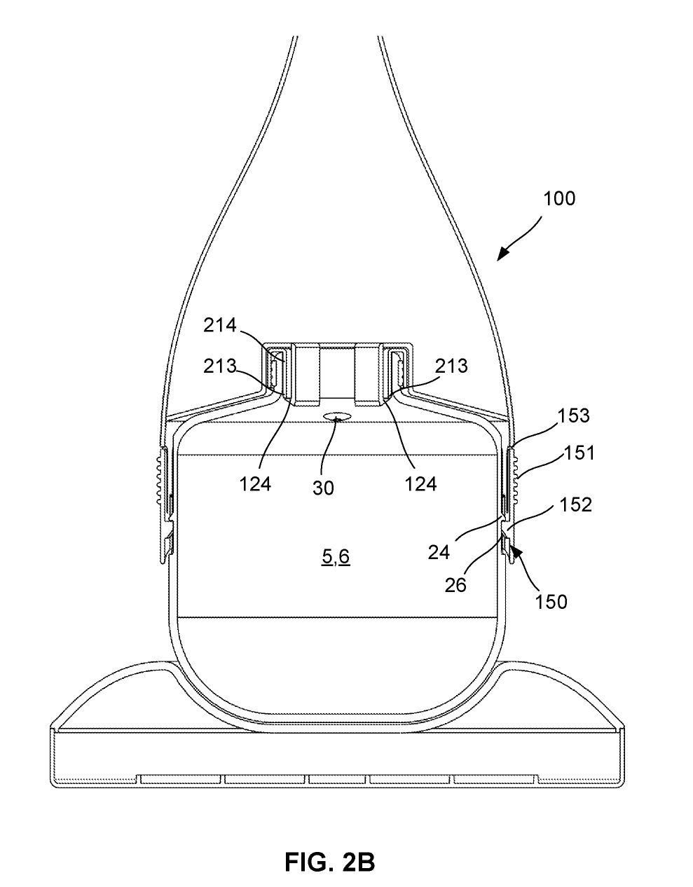

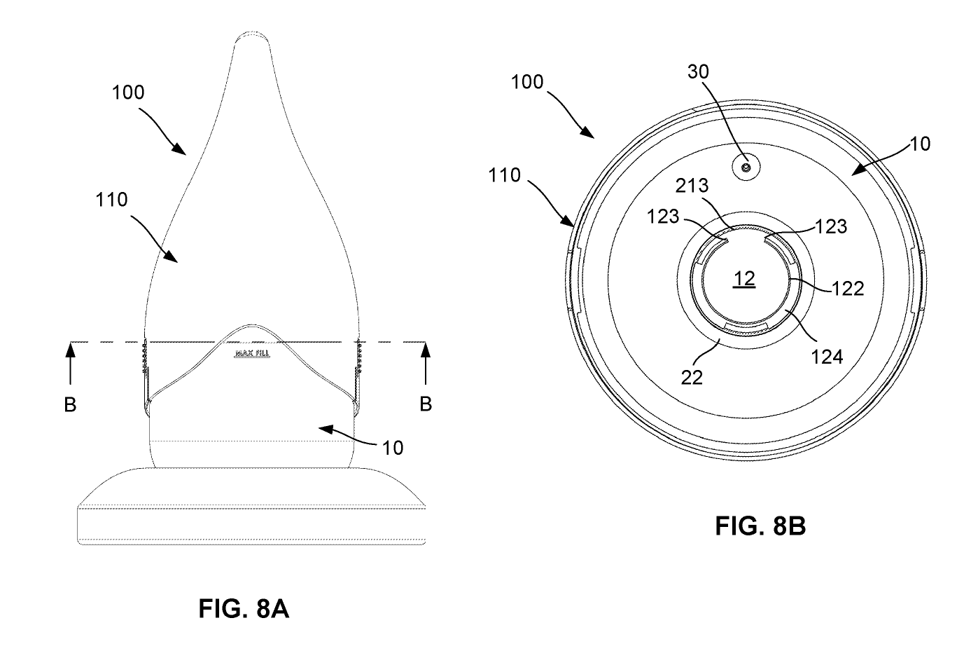

As shown in FIG. 2B and illustrated clearly in FIG. 8B, the tubular element 121 may include one or more resiliently deformable tabs 124 engageable with a bottom surface 213 of the inner wall 214 so as to retain the cap with respect to the fluid delivery attachment 110. As the tubular element 121 is inserted into the cap 200, the tabs 124 will deform slightly so as to allow insertion of the tubular element 121. When the tubular element 121 is fully inserted into the cap 200, the tabs 124 will pass the bottom surface 213 of the inner wall 214 and spring back outward returning to their normal shape. In doing so, the tabs 124 will catch the bottom surface 213 thereby locking the tubular element 121 and thereby the fluid delivery attachment 110 to the cap 200.

The fluid delivery attachment 110 may also be releasably engageable with the vessel 10 as shown in FIGS. 2A and 2B for example. The fluid delivery attachment 110 may have one or more flexible portions 150 each having a grip 151 and a locking tab or projection 152 configured to be received in a recess 26 beneath a catch 24 in the wall of the vessel 10. The flexible portion 150 includes slotted or otherwise weakened regions 153, 154 that allow the flexible portion 150 to pivot substantially about the grip 151 as a subject applies pressure to the grip 151. Accordingly, the fluid delivery attachment 110 is able to be released from the vessel 10 by squeezing the grip 151 which releases the locking tab 152 from the catch 24.

Typically, the locking tabs 124 on the tubular element 121 will locate onto the bottom surface 213 of the inner wall 214 of the cap 200 as the locking tabs 152 on the side of the fluid delivery attachment 110 locate beneath the catches 24 on the side of the vessel 10.

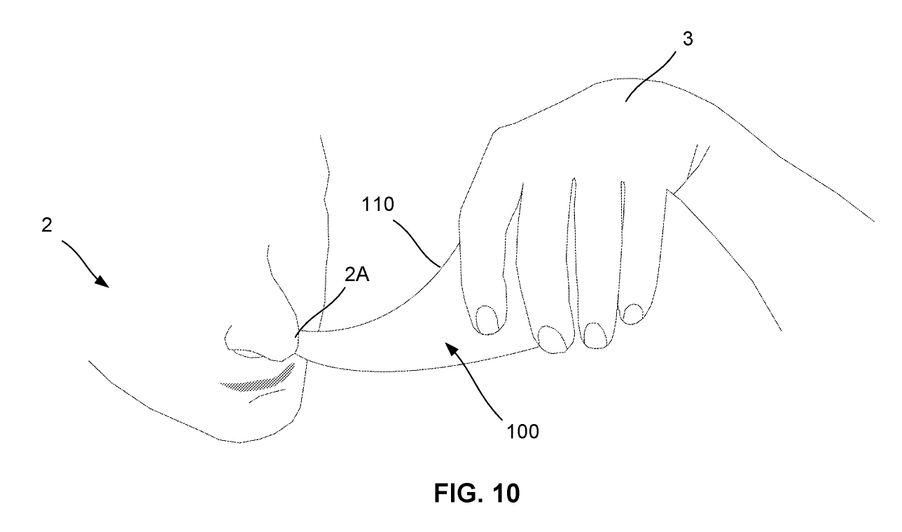

When fully assembled, the apparatus 100 may then be shaken in order to mix the water 5 and additive 6 together to form an irrigation fluid for example. The apparatus 100 is then ready for use. An example of the apparatus 100 being used for nasal irrigation is schematically illustrated in FIG. 10. A subject 2 grips the vessel 10 and/or fluid delivery attachment 110 with their hand 3 and tilts the apparatus in a downward direction to allow the irrigation fluid to flow under gravity from the vessel 10, into the fluid delivery attachment 110 where it is expelled from the fluid outlet 114 into the nasal cavity of the subject 2.

The fluid delivery attachment shown in FIG. 10 is an irrigation attachment 110 having a nasal adaptor portion 112 (refer FIG. 1A) configured to be inserted into a nostril 2A of the subject 2. The irrigation fluid flows into passages and sinus cavities on one side of the face before draining through the other nostril. The procedure can then be repeated by directing irrigation fluid through the other nostril.

In order to regulate flow of fluid through the fluid outlet 114 during irrigation, the vessel 10 may include an elastomeric one-way valve which selectively allows air into the vessel 10 in order to balance air pressure inside and outside of the vessel 10. An example of such a valve 30 is shown in FIGS. 2B and 8B for example. A suitable valve may include an umbrella valve which will equalise air pressure inside and outside of the vessel during irrigation to assist in maintaining a regular flow rate.

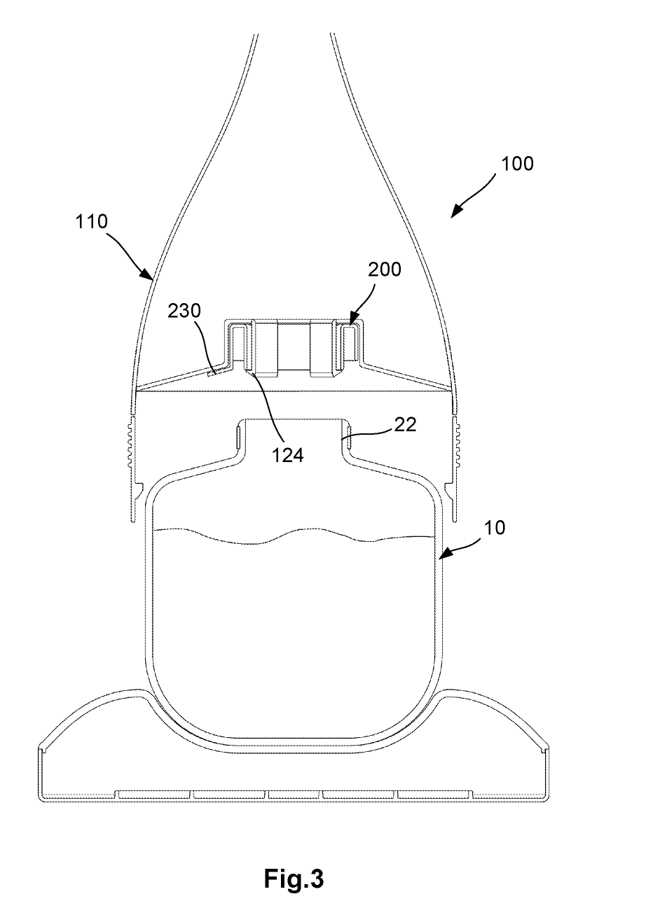

After the irrigation is complete, the fluid delivery attachment 110 is released from the vessel 10 by squeezing grips 151 of the flexible portions 150 which release the locking tabs 151 from respective catches 24. As the cap 200 is retained in the cap engaging portion 120 by locking tabs 124, the cap 200 will be disengaged from the neck region 22 of the vessel 10 while remaining attached to the fluid delivery attachment 110 as shown in FIG. 3. In order to release the cap 200 from the fluid attachment 110, the cap housing 210 includes a grippable release element 230 that is able to be pulled in order to release or tear away the cap 200 from the fluid delivery attachment 110. As shown in FIG. 4, the grippable release element 230 is attached to the bottom of the outer wall 212 of the cap 200. The release element 230 may be a flexible tab or tag that is scored or weakened in the vicinity of its connection to the outer wall 212 enabling it to be gripped by a subject and pulled. The scored or weakened region may further permit the release element 230 to tear the cap 200 apart.

In one example, the release element 230 is pulled downward with sufficient force so that the bottom surface 213 of the inner wall 214 is able to cause the one or more locking tabs 124 to deflect inwardly enough to allow the cap 200 to be released from the cap engaging portion 120. The cap 200 is then left both ruptured and distorted, rendering it unusable and ready to be thrown away or recycled. In alternative arrangements, pulling the release element 230 may cause the cap 200 to tear apart into two or more pieces thereby allowing it to break away from the cap engaging portion 120. Whether the cap 200 is distorted only or physically torn apart, it will be rendered non-reusable by the user, making it a single use cap only which assists in creating a more sterile apparatus 100 for future use.

Referring now to FIGS. 9A and 9B, an example of a steam inhalation apparatus 900 for directing steam into a nasal cavity of a subject is shown. The steam inhalation apparatus 900 uses the same vessel 10 and cap 200 arrangement as previously described. The apparatus 900 includes an inhalation attachment 910 which is releasably engageable with the vessel 10 as previously described.

The inhalation attachment 910 includes a cap engaging portion (not shown) configured to rupture a portion of the cap in order to release an additive from the cap into the water as previously described. The inhalation attachment 910 further includes a mask portion 912 configured to to cover a portion of the subject's face including a nose and mouth of the subject. The mask portion 912 also includes a steam outlet 914 for directing steam into the nasal cavity of the subject. A passageway (internal cavity of inhalation attachment) extends between the cap engaging portion and the steam outlet 914. The inhalation attachment 910 is preferably made from a non-porous plastic such as polycarbonate or other suitable medical grade plastics.

The cap engaging portion is engageable with the cap so as to allow steam to flow between the vessel 10 and the steam outlet 914 when the water is vapourised. The water may be boiled prior to filling the vessel 10 or alternatively the water may be boiled by one or more heating elements disposed in a base portion 50 for example as previously described.

Various fluid delivery attachments may therefore be used with the same vessel and single use cap arrangement as described. The attachments are therefore interchangeable between nasal irrigation and steam inhalation and may be provided in various sizes, subject dependent.

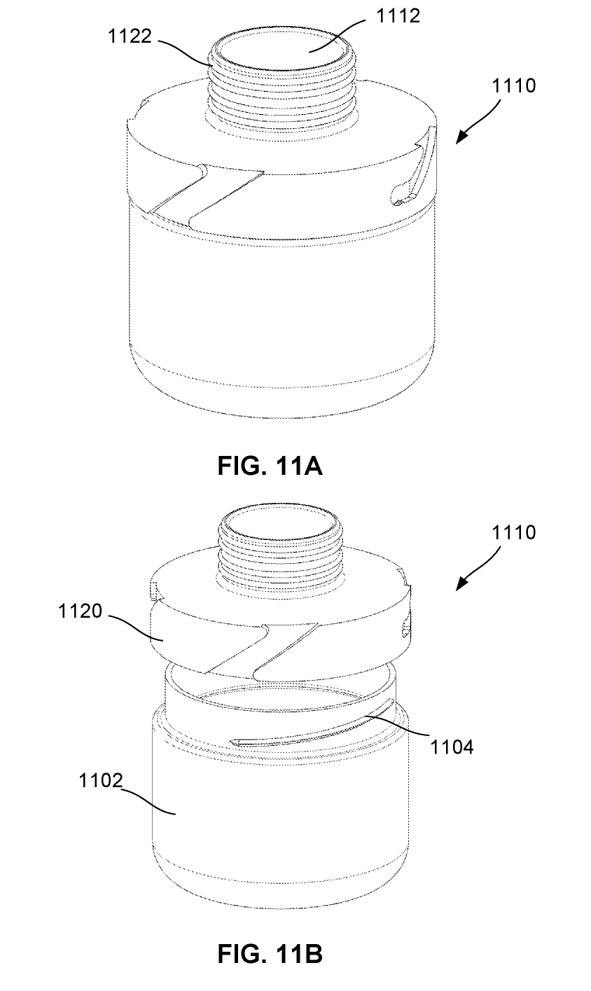

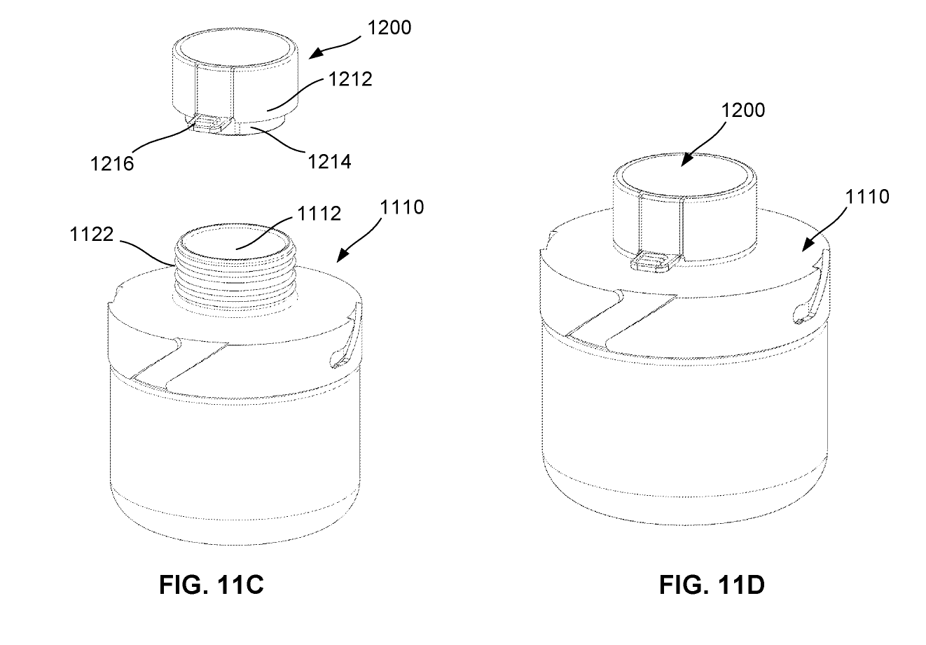

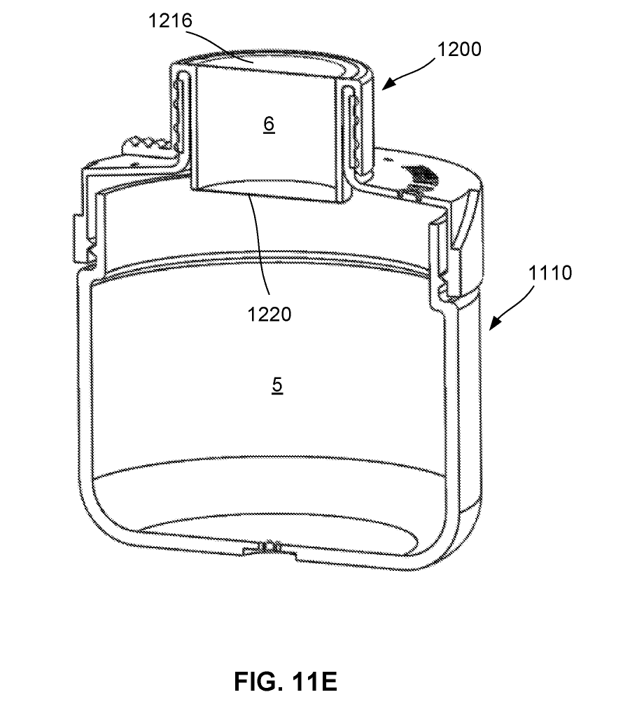

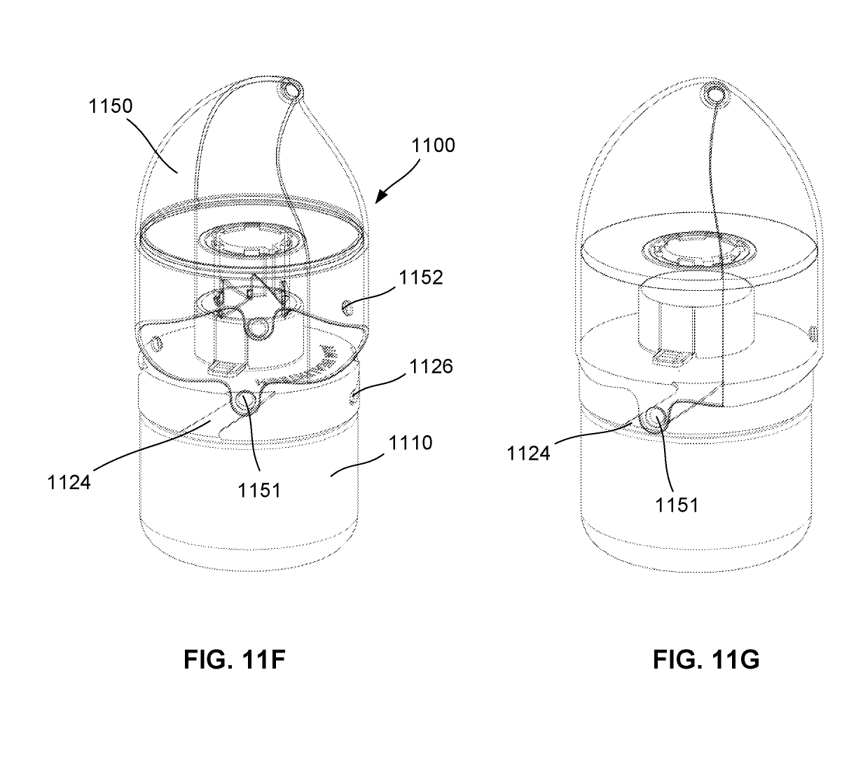

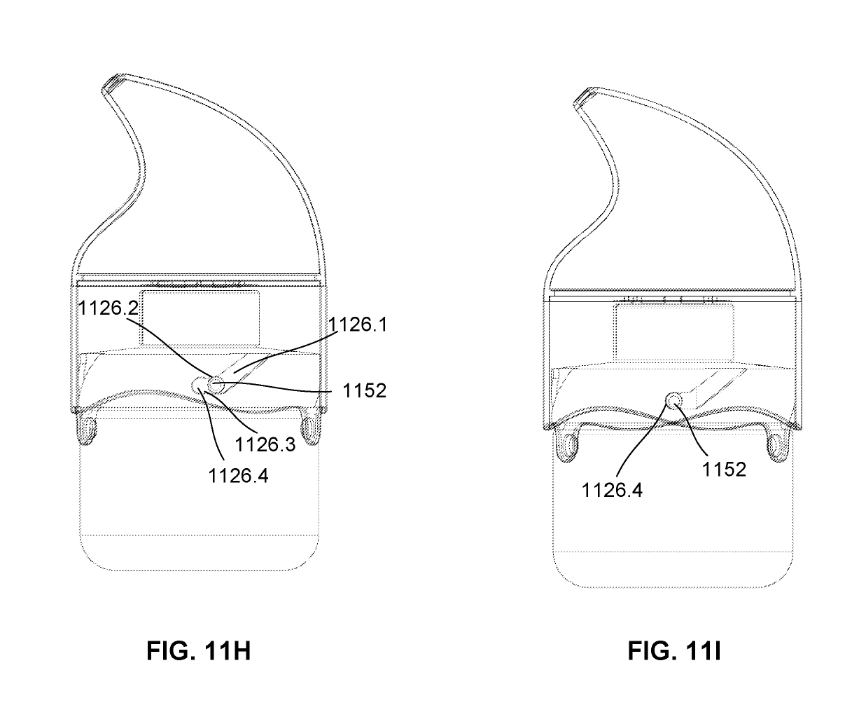

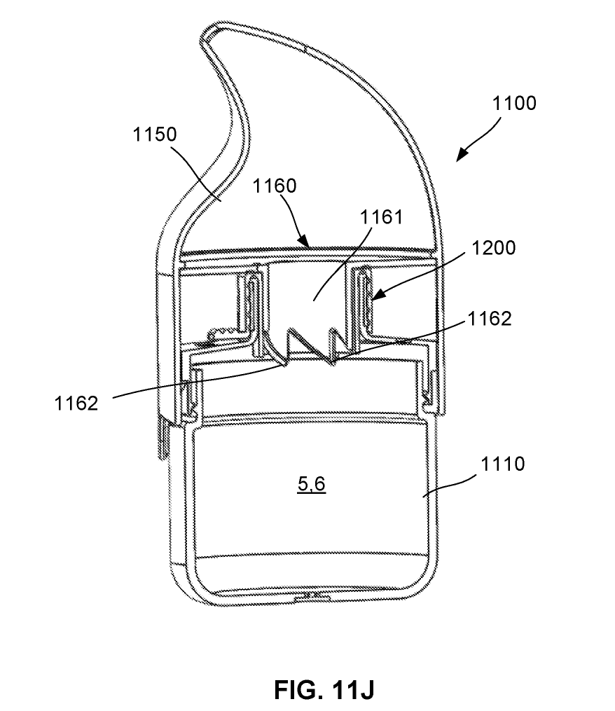

A further example of an irrigation apparatus 1100 for delivering a fluid to a subject will now be described with reference to FIGS. 11A to 11J. In FIGS. 11A to 11B there is shown an alternative vessel 1110 fillable with an amount of water through an opening 1112. The vessel or reservoir 1110 is a two-piece construction including a main body 1102 and a lid 1120 that is threadedly engaged to the body 1102. The lid 1120 is engaged with external thread 1104 disposed on a recessed upper portion of the body and screwed into position. The lid 1120 includes a neck region 1122 that terminates in the vessel opening 1112. A two piece vessel construction promotes easier cleaning of the vessel after use as it is easier to access all internal surfaces of the vessel when disassembled.

A single use cap 1200 for covering the opening and configured to store an additive to be released into the water is shown in FIGS. 11C to 11E. The cap 1200 is pressed onto the vessel 1110 over the neck region 1122 so as to cover the opening 1112. In this respect, the cap 1200 includes an outer wall 1212 spaced apart from an inner wall 1214 and in use, the neck region 1122 of the vessel 1110 is receivable between the inner wall 1214 and the outer wall 1212 to thereby locate the inner wall 1214 within the opening 1112 of the vessel 1110.

As shown most clearly in FIG. 11E, the cap 1200 includes a pair of seals 1216, 1220 disposed proximate the top and bottom of the cap. In one example, each seal is a plastic film that is easily ruptured when the apparatus is used so as to dispense the additive 6 into the water 5. Accordingly, it will be appreciated that in this example, the additive 6 is stored in a cavity inside the cap 1200 between the first and second seals 1216, 1220.

The engagement of the fluid delivery attachment 1150 to the vessel 1110 shall now be described with respect to FIGS. 11F to 11J. The fluid delivery attachment 1150 includes a housing or casing that screws down onto the vessel 1110 and locks into position. The lower profile of the casing includes one or more projecting portions 1151 that are located in grooves 1124 formed around the outer periphery of the vessel lid 1120. As the casing is twisted in a clockwise manner, the projecting portions 1151 follow the respective grooves 1124 as shown in FIG. 11G. As the casing of the fluid delivery attachment begins to engage with the vessel, one or more further projecting portions 1152 in the casing are located in one or more further guide tracks 1126 disposed in the vessel lid 1120 and radially separated from grooves 1124. This is shown in further detail in FIGS. 11H to 11I. Each guide track 1126 may consist of an angled track portion 1126.1 which leads into a locking portion 1126.2. The locking portion may include a necked region 1126.3 which is notched so as to reduce the diameter or cross section of the track. In use, as the casing is being twisted into position, the one or more projecting portions 1152 are forced to follow the guide track 1126 until they reach the notched region 1126.3 which creates resistance to further movement. With the application of suitable force, the projecting portion 1152 may be forced past the notched region 1126.3 and into locking region 1126.4 of the track whereby the attachment is fully bottomed out and locked in position.

In one example, the twist action of the attachment 1150 onto the vessel 1110 is a half turn clockwise twist. In order to ensure continuity throughout the product, the vessel lid 1120 may be attached to the vessel body 1102 also using a half turn clockwise twist. This limited range of motion required to assemble the apparatus is useful for persons with low hand strength and dexterity.

As the attachment 1150 is twisted into position about the vessel, a cap engaging portion 1160 attached to or integral with the attachment 1150, ruptures the cap 1200 to thereby dispense the additive stored therein into the vessel. As shown in FIG. 11J, the cap engaging portion 1160 typically includes a tubular element 1161 that terminates in a longitudinal end having a cutting portion 1162 configured to rupture the cap seal(s). In the example shown, the cutting portion 1162 includes a plurality of serrated teeth configured to pierce the cap as the attachment 1150 is twisted onto the vessel 1110. The top seal or film 1216 is ruptured first followed by the bottom seal 1220 as the cap engaging portion 1160 travels downward through the vessel opening 1112 in a spiral fashion. The progressive rupturing or piercing of the seal as the attachment is located onto the vessel assists in dispensing the additive (e.g. a solution) from the cap into the vessel.

As described previously in respect of earlier examples, the tubular element 1161 of the cap engaging portion 1160 may include one or more resiliently deformable tabs (not shown) engageable with a bottom surface of the inner wall of the cap so as to retain or lock the cap in position with respect to the fluid delivery attachment 1150 during use. After use, the cap 1200 may be torn away from the attachment 1150 for example by pulling a grippable release element or tab 1216 attached to the cap as shown in FIG. 11C.

From the foregoing it should be clear that examples of apparatus described herein for delivering fluid to a subject are capable of having reduced susceptibility to bacterial growth compared to prior art devices, whilst additionally allowing the efficacy, quality and content of the additive to be controlled in a repeatable manner.

Throughout this specification and claims which follow, unless the context requires otherwise, the word “comprise”, and variations such as “comprises” or “comprising”, will be understood to imply the inclusion of a stated integer or group of integers or steps but not the exclusion of any other integer or group of integers.

Persons skilled in the art will appreciate that numerous variations and modifications will become apparent. All such variations and modifications which become apparent to persons skilled in the art, should be considered to fall within the spirit and scope that the invention broadly appearing before described.

The claims defining the invention are as follows:

1) Apparatus for delivering a fluid to a subject, the apparatus including:
a) a vessel fillable with an amount of water through an opening;
b) a cap for covering the opening and configured to store an additive to be released into the water; and
c) a fluid delivery attachment including a body having:
i) a cap engaging portion configured to rupture a portion of the cap in order to release the additive into the water;
ii) a fluid outlet; and
iii) a passageway extending between the cap engaging portion and the fluid outlet,
wherein, the cap engaging portion is engageable with the cap so as to allow fluid to flow between the vessel and the fluid outlet.

2) Apparatus according to claim 1, wherein the cap includes a housing having a top portion containing a weakened region.

3) Apparatus according to claim 2, wherein the additive is stored below the top portion.

4) Apparatus according to claim 3, wherein the additive is stored in the cap by a seal configured to hold the additive.

5) Apparatus according to claim 4, wherein at least part of the cap engaging portion is configured to be inserted into the cap so as to rupture a part of the top portion in the vicinity of the weakened region.

6) Apparatus according to claim 5, wherein rupturing the part of the top portion causes the seal to be ruptured to thereby release the additive into the water.

7) Apparatus according to claim 5 or claim 6, wherein the part of the top portion of the cap that is ruptured remains hingedly connected to the cap allowing it to fold out of the way as the at least part of the cap engaging portion is inserted into the cap.

8) Apparatus according to claim 7, wherein the seal remains hingedly connected to the cap after it has been ruptured, the seal hinging about the same location as the part of the top portion of the cap that is ruptured.

9) Apparatus according to any one of claims 5 to 8, wherein the cap engaging portion includes a tubular element that terminates in a longitudinal end having a cutting portion configured to rupture the part of the top portion.

10) Apparatus according to claim 9, wherein the tubular element is a split tube configured so that a part of the weakened region remains intact when the cutting portion ruptures the part of the top portion.

11) Apparatus according to claim 9 or claim 10, wherein the vessel includes a body having a neck region around the opening.

12) Apparatus according to claim 11, wherein the cap housing includes an outer wall spaced apart from an inner wall that both downwardly depend from the top portion.

13) Apparatus according to claim 12, wherein the neck region of the vessel is receivable between the inner wall and the outer wall to thereby locate the inner wall within the opening of the vessel.

14) Apparatus according to claim 12 or claim 13, wherein the tubular element is positionable within the inner wall to thereby allow fluid to flow from the vessel into the fluid delivery attachment.

15) Apparatus to any one of claims 12 to 14, wherein the tubular element includes one or more resiliently deformable tabs engageable with a bottom surface of the inner wall so as to retain the cap with respect to the fluid delivery attachment.

16) Apparatus according to claim 15, wherein the cap housing further includes a grippable release element that is able to be pulled in order to release or tear the cap away from the fluid delivery attachment.

17) Apparatus according to claim 16, wherein the grippable release element is attached to the outer wall.

18) Apparatus according to claim 17, wherein the grippable release element is a flexible tab.

19) Apparatus according to any one of the preceding claims wherein the cap is for a single use.

20) Apparatus according to any one of the preceding claims, wherein the fluid delivery attachment is releasably engageable with the vessel.

21) Apparatus according to any one of the preceding claims, wherein the vessel is made from glass or a non-porous plastic.

22) Apparatus according to claim 21, wherein the vessel includes an elastomeric one-way valve which selectively allows air into the vessel in order to balance air pressure inside and outside of the vessel in order to regulate flow of fluid through the fluid outlet.

23) Apparatus according to any one of the preceding claims wherein the fluid delivery attachment is made from a non-porous plastic.

24) Apparatus according to any one of the preceding claims wherein the additive is a dissolvable tablet.

25) Apparatus according to any one of claims 1 to 23, wherein the additive is a liquid.

26) Apparatus according to claim 24 or claim 25, wherein the additive contains a saline composition.

27) Apparatus according to claim 24 or claim 25, wherein the additive contains a fragrance or medicament.

28) Apparatus according to any one of claims 1 to 26, wherein the fluid delivery attachment is for nasal irrigation.

29) Apparatus according to claim 27, wherein the fluid delivery attachment is for steam inhalation.

30) Apparatus according to any one of the preceding claims, further including a base portion configured to receive the vessel.

31) Apparatus according to claim 30, wherein the base portion is configured to heat the vessel.

32) Apparatus according to claim 31, wherein the base portion includes one or more inductive heating elements.

33) Apparatus according to claim 1, wherein the cap includes a housing having a first seal located at the top of housing and second seal located at the bottom of the housing, and wherein the additive is stored in a cavity between the first and second seals.

34) Apparatus according to claim 33 wherein at least part of the cap engaging portion is configured to be inserted into the cap so as to rupture a part of the first and second seals to thereby cause the additive to be released into the water.

35) Apparatus according to claim 1, wherein the vessel is a two-part construction including a body portion releasably engageable with a lid portion, the opening disposed in the lid portion.

36) A nasal irrigation apparatus for irrigating a nasal cavity of a subject, the apparatus including:
a) a vessel fillable with an amount of water through an opening;
b) a cap for covering the opening and configured to store an additive to be released into the water; and
c) an irrigation attachment, including a body having:
i) a cap engaging portion configured to rupture a portion of the cap in order to release the additive into the water to form an irrigation fluid;
ii) a nasal adaptor portion configured to be inserted into a nostril of the subject, said nasal adaptor portion terminating in a fluid outlet for expelling said irrigation fluid into the nasal cavity; and
iii) a passageway extending between the cap engaging portion and the fluid outlet,
wherein, the cap engaging portion is engageable with the cap so as to allow irrigation fluid to flow between the vessel and the fluid outlet.

37) A steam inhalation apparatus for directing steam into a nasal cavity of a subject, the apparatus including:
a) a vessel fillable with an amount of water through an opening;
b) a cap for covering the opening and configured to store an additive to be released into the water; and
c) an inhalation attachment, including a body having:
i) a cap engaging portion configured to rupture a portion of the cap in order to release the additive into the water; and
ii) a mask portion configured to cover a portion of the subject's face including a nose and mouth of the subject, the mask portion including a steam outlet for directing steam into the nasal cavity; and
iii) a passageway extending between the cap engaging portion and the steam outlet,
wherein, the cap engaging portion is engageable with the cap so as to allow steam to flow between the vessel and the steam outlet when the water is vapourised.

38) A cap for releasing an additive into an amount of water in a vessel, the cap including:
a) a housing engageable with the vessel to cover an opening thereof, the housing configured to store the additive, the housing including:
i) a rupturable portion configured to be ruptured when a fluid delivery attachment for delivering fluid to a subject is engaged with the housing to thereby cause the additive to be released into the water,
wherein, the rupturable portion is rupturable so as to allow at least part of the fluid delivery attachment to be inserted into the housing in the vicinity of the opening of the vessel to thereby allow fluid to flow from the vessel to the fluid delivery attachment whereby it is deliverable to the subject.

39) A cap according to claim 37, wherein the rupturable portion contains a weakened region configured to be ruptured by the fluid delivery attachment.

40) A cap according to claim 38, wherein the additive is stored below the rupturable portion.

41) A cap according to claim 39, wherein the additive is held in the housing by a seal.

42) A cap according to claim 40, wherein rupturing the rupturable portion causes the seal to be ruptured to thereby release the additive into the water.

43) A cap according to claim 40 or claim 41, wherein the rupturable portion remains hingedly connected to the housing after it has been ruptured so as to allow it to fold out of the way as the at least part of the fluid delivery attachment is inserted into the housing.

44) A cap according to claim 42, wherein the seal remains hingedly connected to the housing after it has been ruptured, the seal hinging about the same location as the rupturable portion.

45) A cap according to any one of claims 37 to 43, wherein the housing includes:
a) an inner wall; and
b) an outer wall spaced apart from the inner wall to thereby define an opening receivable of a neck region of the vessel.

46) A cap according to claim 44, wherein the housing further includes a top portion from which the inner wall and the outer wall downwardly depend.

47) A cap according to claim 45, wherein at least part of the top portion forms the rupturable portion.

48) A cap according to any one of claims 44 to 46, wherein the housing further includes a grippable release element that is able to be pulled in order to release or tear the cap away from the fluid delivery attachment.

49) A cap according to claim 47, wherein the grippable release element is attached to the outer wall.

50) A cap according to claim 47 or claim 48, wherein the grippable release element is a flexible tab.

51) A cap according to any one of claims 37 to 48, wherein the cap is for a single use.

52) A cap according to any one of claims 37 to 50, wherein the additive includes at least one of:
a) a saline composition;
b) a medicament; and
c) a fragrance.

53) A cap according to claim 51, wherein the additive is either in solid or liquid form.

54) A cap according to claim 37, wherein the rupturable portion includes a first seal located at the top of the cap housing and second seal located at the bottom of the cap housing, the additive stored in a cavity between the first and second seals.

55) A cap according to claim 53, wherein the first and second seals are plastic films.
